(19) United States
(12) Patent Application Publication
(10) Pub. No.: US 2024/0197248 A1
(43) Pub. Date: Jun. 20, 2024

(54) BIOMETRIC INFORMATION MEASUREMENT DEVICE, CONTROL METHOD OF BIOMETRIC INFORMATION MEASUREMENT DEVICE, AND PROGRAM

(71) Applicant: OMRON HEALTHCARE CO., LTD, Kyoto (JP)

(72) Inventors: Takahiro HAMAGUCHI, Kyoto (JP); Yuka TANABE, Kyoto (JP); Kentaro MORI, Kyoto (JP); Takeshi KUBO, Kyoto (JP); Yuki TAKANO, Kyoto (JP); Yasuo ASANO, Kyoto (JP); Rika KATO, Kyoto (JP)

(73) Assignee: OMRON HEALTHCARE CO., LTD, Kyoto (JP)

(21) Appl. No.: 18/592,577

(22) Filed: Mar. 1, 2024

Related U.S. Application Data
(63) Continuation of application No. PCT/JP2023/004636, filed on Feb. 10, 2023.

(30) Foreign Application Priority Data
Jun. 23, 2022 (JP) 2022-101234

(51) Int. Cl. A61B 5/00; A61B 5/022; A61B 5/256; A61B 5/28

(52) U.S. Cl. CPC A61B 5/681; A61B 5/02233; A61B 5/256; A61B 5/28; A61B 5/6844

(57) ABSTRACT

This biometric information measurement device is used while attached to a wrist of a human body, and comprises: a blood pressure measurement unit; an electrocardiographic waveform measurement unit provided with a plurality of electrodes and is for measuring electrocardiographic waveforms of the human body; an electrode contact state detection unit that detects the contact states of the human body with the plurality of electrodes; a position detection unit; a controller; a first determining unit that determines whether the device is positioned at a height within a predetermined range; a second determining unit that determines whether the human body is stably in contact with the plurality of electrodes; and a batch measurement controller that performs control for executing, in batches, the blood pressure measurement by unit of the blood pressure measurement unit and the measurement of the electrocardiographic waveforms of the human body by unit of the electrocardiographic waveform measurement unit.

### CROSS-REFERENCE TO RELATED APPLICATIONS

This application is the U.S. national stage application filed pursuant to 35 U.S.C. 365(c) and 120 as a continuation of International Patent Application No. PCT/JP2023/004636, filed Feb. 10, 2023, which application claims priority to Japanese Patent Application No. 2022-101234, filed Jun. 23, 2022, which applications are incorporated herein by reference in their entireties.

### TECHNICAL FIELD

The present invention belongs to a technical field related to healthcare, and particularly relates to a biological information measurement device, a control method of a biological information measurement device, and a program.

### BACKGROUND ART

In recent years, it has been common for an individual to measure his/her body/health information (hereinafter, also referred to as biological information) such as a blood pressure value and an electrocardiographic waveform by using a measurement instrument on a daily basis and utilize the measurement result for health management. For this reason, there is an increasing demand for instruments focusing on portability, many portable measurement devices have been proposed, and a portable instrument capable of measuring both the blood pressure value and the electrocardiographic waveform has also been proposed (for example, Patent Literature 1 and the like).

Patent Literature 1 discloses a portable electrocardiogram measurement device having unit for performing blood pressure measurement in an electrocardiographic waveform measurement device worn on a wrist of a human body by using a belt including an electrode. According to the present invention, by carrying the device, a user can obtain electrocardiographic waveform information indicating the electrical activity of a heart and measure the blood pressure at an arbitrary timing such as when the user feels a pain in a chest. In addition, it is also described that, when the device is worn on the wrist, the electrocardiographic waveform (and the blood pressure) can be measured by so-called IV induction only by placing, on the chest, a device body fixed to an arm, and the electrocardiographic waveform can be measured by I induction by attaching the device to one (right) arm and touching the electrode arranged on the device body with the other (left) hand. According to these measurement methods, it is not necessary to hold the device body with a hand, and unnecessary force is not applied, so that a signal with less noise caused by myoelectric potential or the like can be obtained.

### CITATION LIST

### Patent Literature

Patent Literature 1: JP 2007-195693 A

### SUMMARY OF INVENTION

By the way, according to the technique described in Patent Literature 1, when performing measurement using the device, in the case of IV induction, the user wears the device on the right wrist and then brings the electrode into contact with the chest, and presses the measurement start button of the device body with the left hand on which the device is not worn, to start the measurement. In addition, in the case of I induction, it is necessary to press the measurement start button with the left hand and then touch the electrode of the device body with the hand.

However, in such a measurement mode, there is a possibility that a state where a contact state with the electrode is not appropriate occurs after the measurement start button is pressed (that is, after the measurement is started), so that an unstable electrocardiogram may be recorded, or that particularly in the case of I induction, the measurement proceeds without touching the electrode. An explanatory diagram illustrating such a situation is illustrated in FIG. 5. In addition, in a case where the blood pressure measurement is performed together with the measurement of the electrocardiographic waveform, it is necessary to take a posture in which the device body (a place where the blood pressure measurement is performed) is arranged at the same height as the height of the heart, so that the user is forced to take the inconvenient posture, which makes the above problem more remarkable.

In view of the above problems, an object of the present invention is to provide a technique for accurately measuring blood pressure and an electrocardiographic waveform in a portable biological information measurement device capable of measuring the blood pressure and the electrocardiographic waveform.

In order to solve the above problem, the present invention adopts the following configuration. That is, provided is a biological information measurement device used by being worn on a wrist of a human body, the biological information measurement device including:
a blood pressure measurement unit configured to measure a blood pressure of the human body;
an electrocardiographic waveform measurement unit which includes a plurality of electrodes and is configured to measure an electrocardiographic waveform of the human body;
an electrode contact state detection unit configured to detect a contact state of the human body with the plurality of electrodes;
a position detection unit configured to detect a position of the device; and
a controller which controls the electrocardiographic waveform measurement unit and the blood pressure measurement unit,
in which the controller includes
   a first correctness determination unit which determines, on the basis of an output of the position detection unit, correctness as to whether or not a wrist of the human body on which the device is worn is positioned at a height within a predetermined range,
   a second correctness determination unit which determines, on the basis of an output of the electrode contact state detection unit, whether or not the human body is stably in contact with the plurality of electrodes, and
   a collective measurement control unit which performs control to collectively execute blood pressure measurement of the human body by the blood pressure measurement unit and measurement of an electrocardiographic waveform of the human body by the electrocardiographic waveform measurement unit, on condition that at least a determination result of the second correctness determination unit indicates correctness.

Note that in the present specification, “measurement of an electrocardiographic waveform” unit recording of waveform data of an electrocardiographic signal. In addition, “collectively” includes simultaneous and parallel execution. In addition, examples of the above-described blood pressure measurement unit include a cuff, a pressure sensor, a pump, and the like for measuring blood pressure by an oscillometric method, but the present invention is not limited thereto. In addition, for example, a triaxial acceleration sensor can be adopted as the position detection unit, but other unit may be used as long as at least the position of the device on the vertical axis (that is, the height at which the device is positioned) can be detected.

According to such a configuration, the measurement of the blood pressure and the electrocardiographic waveform can be collectively performed on condition that an electrode contact state suitable for the measurement of the electrocardiographic waveform is obtained (without requiring any measurement start operation). Therefore, the electrocardiographic waveform is not measured in a state where the electrocardiographic waveform is not correctly in contact with the electrode for electrocardiographic waveform measurement, the measurement does not include the noise caused by the operation for correcting the posture immediately after the start of the measurement, and two types of biological information of the blood pressure value and the electrocardiographic waveform can be collectively measured accurately and easily.

In addition, on condition that the first correctness determination unit outputs a determination result of the correctness, the second correctness determination unit may determine correctness as to whether or not the human body is stably in contact with the plurality of electrodes. That is, the collective measurement may be performed only in a case where determination result of the first correctness determination unit and the second correctness determination unit indicate correctness. Under such conditions, the collective measurement is performed on condition that a state is appropriate for the measurement of the blood pressure and the electrocardiographic waveform, and thus, the blood pressure value and the electrocardiographic waveform with high accuracy can be acquired.

Note that, in a measurement device of a wrist-worn type, when a posture of being in contact with a plurality of electrodes is first taken for the electrocardiographic waveform measurement, the posture easily becomes unnatural when the height of the device is adjusted for the blood pressure measurement thereafter. In such an unnatural posture, it is difficult to stably contact the electrode, or an unnecessary force is applied, so that myoelectric potential (noise) is likely to be mixed. In this regard, with the configuration as described above, the correctness regarding the height is determined first, and thus the measurement in an unnatural posture can be suppressed.

Note that a height within the predetermined range may be set to be substantially same as a height of a heart of the human body. Such a height is suitable for the blood pressure measurement. In addition, the biological information measurement device may be a wristwatch-type wearable device.

In addition, the present invention can also be regarded as a control method of a device as follow. That is, provided is a control method of a biological information measurement device which is used by being worn on a wrist of a human body and includes
a blood pressure measurement unit configured to measure a blood pressure of the human body,
an electrocardiographic waveform measurement unit which includes a plurality of electrodes and is configured to measure an electrocardiographic waveform of the human body,
an electrode contact state detection unit configured to detects a contact state of the human body with the plurality of electrodes, and
a position detection unit configured to detects a position of the device, the control method including:
a first correctness determination step which determines, on the basis of an output of the position detection unit, correctness as to whether or not a wrist of the human body on which the device is worn is positioned at a height within a predetermined range;
a second correctness determination step which determines, on the basis of an output of the electrode contact state detection unit, correctness as to whether or not the human body is stably in contact with the plurality of electrodes; and
a collective measurement step which collectively executes blood pressure measurement of the human body by the blood pressure measurement unit and measurement of an electrocardiographic waveform of the human body by the electrocardiographic waveform measurement unit, in a case where a determination result of the second correctness determination step indicates correctness.

In addition, the second correctness determination step may be executed after the first correctness determination step. In addition, a height within the predetermined range may be set to be substantially same as a height of a heart of the human body.

In addition, the present invention can also be regarded as a program for causing the biological information measurement device to execute the above-described method, and a computer-readable recording medium in which such a program is non-transitory recorded.

Note that each of the above-described configurations can be combined with each other to constitute the present invention as long as no technical contradiction occurs.

According to the present invention, it is possible to provide a technique for accurately measuring blood pressure and an electrocardiographic waveform in a portable biological information measurement device capable of measuring the blood pressure and the electrocardiographic waveform.

### BRIEF DESCRIPTION OF DRAWINGS

FIG. 1A is a schematic view illustrating an appearance of a biological information measurement device according to a first embodiment. FIG. 1B is an explanatory view illustrating a state when the biological information measurement device of the first embodiment is worn.

FIG. 2 is a functional block diagram illustrating a functional configuration of the biological information measurement device according to the first embodiment.

FIG. 3A is a first view illustrating an example of an image output in the biological information measurement device of the first embodiment. FIG. 3B is a second view illustrating an example of an image output in the biological information measurement device of the first embodiment. FIG. 3C is a third view illustrating an example of an image output in the biological information measurement device of the first embodiment. FIG. 3D is a fourth view illustrating an example of an image output in the biological information measurement device of the first embodiment.

FIG. 4 is a flowchart illustrating a part of processing performed in the biological information measurement device of the first embodiment.

FIG. 5 is an explanatory diagram for explaining a problem in the conventional technique.

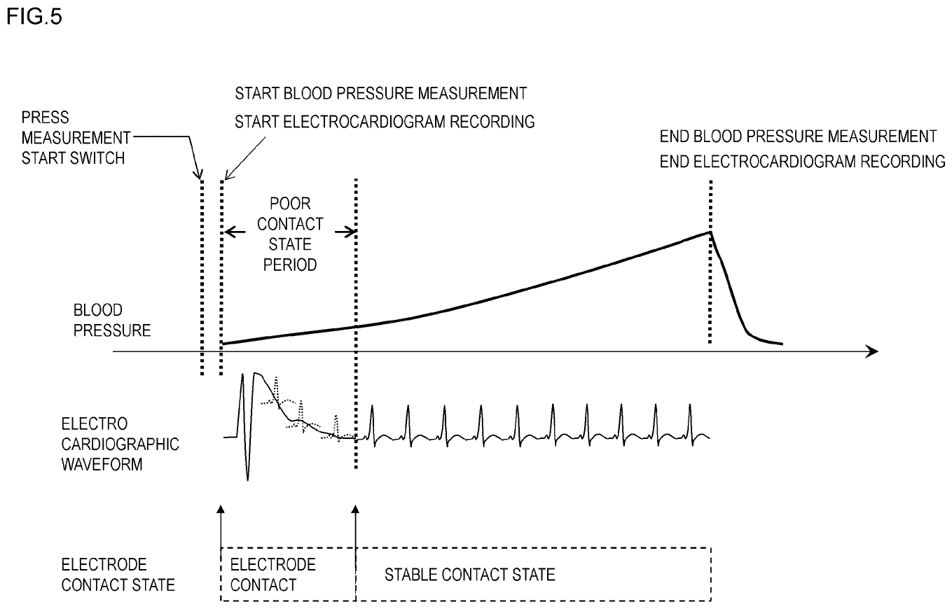

### DESCRIPTION OF EMBODIMENTS

### First Embodiment

Hereinafter, specific embodiments of the present invention will be described on the basis of the drawings. However, the dimensions, materials, shapes, relative arrangements, and the like of the components described in this embodiment are not intended to limit the scope of the present invention only to them unless otherwise specified.

### (Overall Configuration of Device)

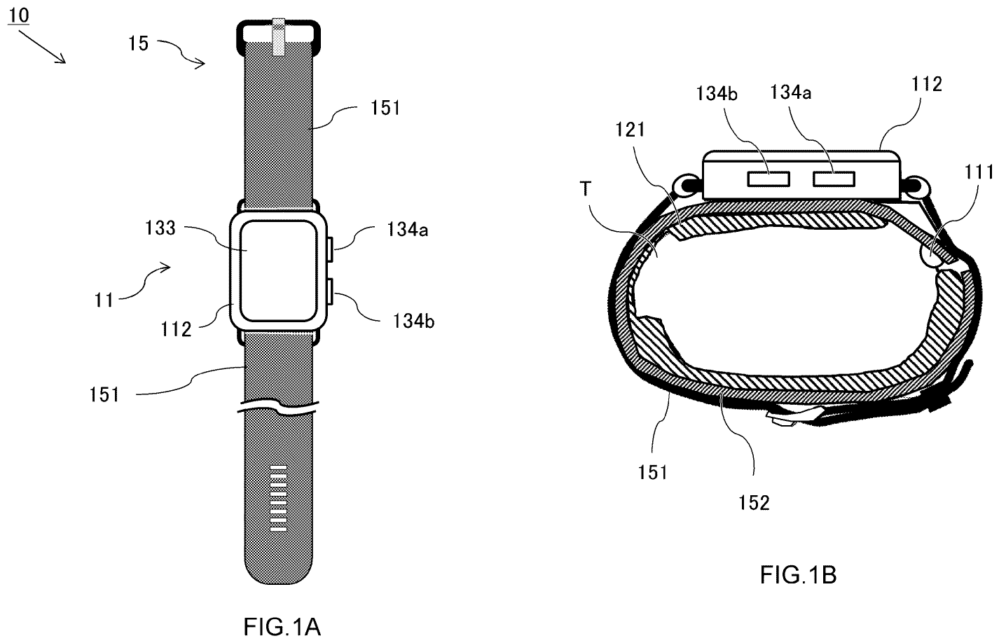

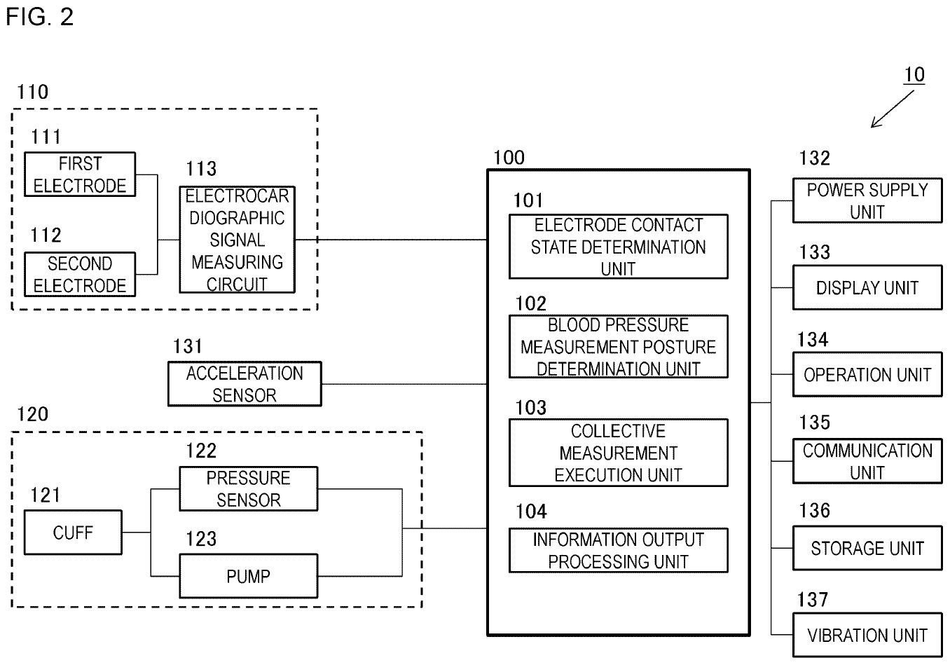

FIG. 1A is a schematic view illustrating an external configuration of a biological information measurement device 10 according to the present embodiment. FIG. 1B is an explanatory view illustrating a state when the biological information measurement device 10 according to the present embodiment is worn on a wrist T FIG. 2 is a functional block diagram illustrating a functional configuration of the biological information measurement device 10 according to the present embodiment.

As illustrated in FIGS. 1A, 1B, and 2, the biological information measurement device 10 is schematically a wristwatch-type wearable device including a main body 11 and a belt portion 15, and can measure a blood pressure value and an electrocardiographic waveform in the state of being worn on the wrist T of a human body.

The main body 11 includes a display unit 133 (for example, a liquid crystal display or the like can be adopted), operation buttons 134a, 134b, a bezel functioning as second electrode 112, an acceleration sensor 131, and the like. The acceleration sensor 131 corresponds to position detection unit according to the present invention, and detects the position/posture of the biological information measurement device 10. In addition, as illustrated in FIG. 2, the main body 11 includes a control unit 100, an electrocardiographic signal measurement unit 110, a blood pressure measurement unit 120, a power supply unit 132, a display unit 133, an operation unit 134, a communication unit 135, a storage unit 136, and a vibration unit 137 as functional configurations thereof.

These functional configurations will be described later.

In addition, the belt portion 15 includes a cuff 121 for compressing an artery in the wrist T, a curler 152 for supporting the cuff 121, a first electrode 111, and a belt 151 for fixing the biological information measurement device 10 to the wrist T. For example, the belt 151 includes a parent-side band and a tip-side band, and can adopt a shape of a type in which the tip-side band is fixed by a buckle of the parent-side band. However, the belt may have any configuration as long as the biological information measurement device 10 can be appropriately fixed to the wrist T. For example, it is also possible to adopt a configuration in which fixing is performed by a hook-and-loop fastener.

### (Functional Configurations of Main Body)

Next, the functional configurations of the main body 11 will be described. The control unit 100 controls the entire biological information measurement device 10 including the electrocardiographic signal measurement unit 110, the blood pressure measurement unit 120, and the like. In addition, the control unit 100 includes functional units of an electrode contact state determination unit 101, a blood pressure measurement posture determination unit 102, a collective measurement execution unit 103, and an information output processing unit 104, and reads and executes a program from the storage unit 136 described later, thereby controlling each configuration of the biological information measurement device 10 to realize the functional units which fulfill these predetermined purposes. Note that the control unit 100 includes a processor such as a central processing unit (CPU) in terms of hardware.

The electrocardiographic signal measurement unit 110 includes the first electrode 111, the second electrode 112, and an electrocardiographic signal measuring circuit 113, and measures an electrocardiographic signal of a user on the basis of a potential difference between the first electrode 111 and the second electrode 112 in contact with a human body surface (specifically, the wrist of one hand and the finger of the other hand) (by so-called I induction). In addition, the electrocardiographic signal measuring circuit 113 also detects a contact state of the skin surface of the user with the first electrode 111 and the second electrode 112. That is, the electrocardiographic signal measuring circuit 113 in the present embodiment also serves as electrode contact state detection unit according to the present invention. Note that in addition, the electrocardiographic signal measurement unit 110 also includes an AD conversion circuit, an amplifier, a filter, and the like (not illustrated), but since these are configured by known techniques, the description thereof is omitted.

The blood pressure measurement unit 120 includes the cuff 121, the pressure sensor 122, and a pump 123, and measures the blood pressure of the user by a so-called oscillometric method. The blood pressure measurement by the oscillometric method is a well-known technique, and thus a detailed description thereof will be omitted.

The power supply unit 132 includes a battery (not illustrated) which supplies power necessary for operating the device. For example, the battery may be a secondary battery such as a lithium ion battery, or may be a primary battery.

The display unit 133 includes a display device such as a liquid crystal display, and displays, on the display device, various types of information including guide information regarding the operation of the device. Note that the display unit 133 may further include an LED indicator or the like. In addition, the operation unit 134 includes operation buttons 134a and 134b, and receives the input operation of the user via these buttons. Note that the operation unit 134 can also receive an input of a user operation by receiving an input signal from another electronic instrument via the communication unit 135 described later.

The communication unit 135 includes an antenna (not illustrated) for wireless communication, and performs information communication with another electronic instrument such as an information processing terminal by, for example, BLE communication. Note that a terminal for wired communication may be provided.

The storage unit 136 includes a main storage device (not illustrated) such as a random access memory (RAM), and stores various types of information such as an application program, a measured electrocardiographic waveform, a blood pressure, and guide information. In addition to the RAM, a long-term storage medium such as a flash memory may be provided. In addition, electrocardiographic waveform data, a measured blood pressure value, and the like is stored.

The vibration unit 137 includes a vibrator (not illustrated) including a small motor or the like, and generates vibration in a predetermined pattern set for each guidance content. Accordingly, it is possible to notify the user of predetermined guidance information corresponding to the pattern.

Next, each functional unit included in the control unit 100 will be described. The electrode contact state determination unit 101 determines, on the basis of the output of the electrocardiographic signal measuring circuit 113, whether or not the user is stably in contact with the first electrode 111 and the second electrode 112. Whether or not the contact is stable can be distinguished by an arbitrary index, but for example, evaluation may be performed by using information such as the baseline fluctuation of the electrocardiographic waveform and the posture fluctuation of the device based on the output of the acceleration sensor 131.

On the basis of the output of the acceleration sensor 131, the blood pressure measurement posture determination unit 102 determines correctness as to whether or not the wrist of the user in the state of wearing the device is positioned at a height within a predetermined range, more specifically, whether or not the wrist is positioned at a height substantially equal to the height of the heart. In addition, it may be determined whether or not the height is continuously maintained.

On the basis of the outputs of the electrode contact state determination unit 101 and the blood pressure measurement posture determination unit 102, the collective measurement execution unit 103 performs control to collectively execute the measurement of the blood pressure by the blood pressure measurement unit 120 and the measurement of the electrocardiographic waveform in a case where these determination results are both correct. Note that here, the measurement of the electrocardiographic waveform unit recording, as waveform data, the electrocardiographic signal measured by the electrocardiographic signal measurement unit 110. That is, in the present embodiment, electrocardiographic waveform measurement unit includes the electrocardiographic signal measurement unit 110 and the storage unit 136.

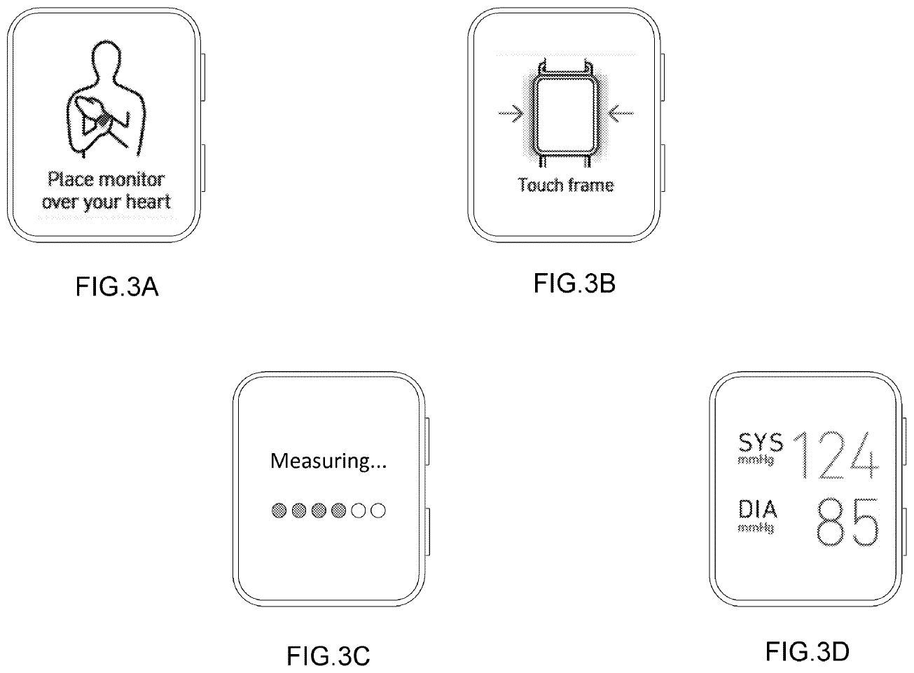

The information output processing unit 104 outputs guide information regarding the use of the device by image display by the display unit 133 and a vibration pattern by the vibration unit 137. Specifically, for example, control of outputting information for guiding a posture for measuring biological information to the user, information for guiding each of the start and end of measurement, and the like is executed. FIGS. 3A to 3D illustrate examples of guide images displayed on the display unit 133.

FIG. 3A is a guide image for guiding raising, to the height of the heart, the wrist on which the device is worn and maintaining the wrist in preparation for measurement. FIG. 3B is a guide image for guiding touching the second electrode 112 of the device in preparation for measurement. FIG. 3C is a guide image for guiding that blood pressure (electrocardiogram) measurement is being performed. FIG. 3D is a guide image illustrating a measurement result after the end of the measurement. Note that each image may be a still image or may be a moving image.

### (Biological Information Measurement Processing)

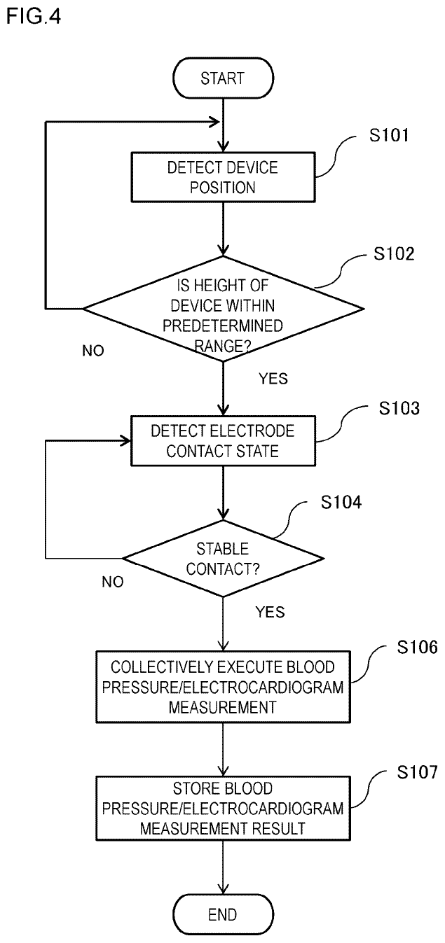

Next, a flow of processing when the biological information measurement device 10 executes measurement of the biological information will be described on the basis of FIG. 4. FIG. 4 is a flowchart illustrating a procedure of processing when the biological information measurement device 10 according to the present embodiment is used to collectively measure the blood pressure and the electrocardiographic waveform.

First, when the biological information measurement device 10 is powered on, the acceleration sensor 131 detects the position/posture of the device (S101), and the blood pressure measurement posture determination unit 102 determines, on the basis of on the output of the acceleration sensor 131, whether or not the height of the biological information measurement device 10 is within a predetermined range (S102). Here, in a case where it is determined that the height of the device is not within the predetermined range, the process returns to step S101, and the determination processing as to whether the height of the device is within the predetermined range is repeated on the basis of the output of the acceleration sensor 131.

On the other hand, in a case where it is determined in step S102 that the height of the device is within the predetermined range, the process proceeds to step S103. In step S103, the electrocardiographic signal measuring circuit 113 detects a contact state of the human body (user) with the first electrode 111 and the second electrode 112 (S103). Then, the electrode contact state determination unit 101 determines, on the basis of the output of the electrocardiographic signal measuring circuit 113, correctness as to whether or not the user is stably in contact with the first electrode 111 and the second electrode 112 (S104). Here, in a case where it is determined that the user is not stably in contact with each electrode, the process returns to step S103, and the subsequent processing is repeated.

On the other hand, when it is determined in step S104 that the user is stably in contact with each electrode, the collective measurement execution unit 103 performs control to collectively execute the measurement of the blood pressure and the measurement of the electrocardiographic waveform by the blood pressure measurement unit 120 (S105). Then, when the measurement of the blood pressure is ended, the measurement of the electrocardiographic waveform (that is, recording of waveform data) is also ended simultaneously, the measurement result is stored in the storage unit 136 (S106), and this routine is temporarily ended.

Note that the information output processing unit 104 may output the guide information at an appropriate timing of the above flow. For example, prior to step S101, a guide that the wrist on which the device is worn is to be raised to the height of the heart and maintained may be provided by displaying the guide image illustrated in FIG. 3A on the display unit 133 or by the vibration of the vibration unit 137. In addition, after step S106, an image (see FIG. 3D) indicating the measurement result may be displayed on the display unit 133.

According to the biological information measurement device 10 according to the present embodiment as described above, the blood pressure and the electrocardiographic waveform are collectively measured by maintaining, at a height suitable for blood pressure measurement, a site where the blood pressure is measured in the state where the device is worn (that is, the position of the device) and establishing a situation of the stable contact with the electrode. Therefore, it is possible to prevent the measurement from being performed in an inappropriate posture or situation for performing the measurement, and it is possible to obtain a measurement result with high accuracy for both the blood pressure and the electrocardiographic waveform. In addition, in the biological information measurement device 10 according to the present embodiment, when the power is turned on, determination as to whether or not the wrist of the user is positioned at a height within a predetermined range and determination as to whether or not the user is stably in contact with each electrode are automatically performed, and thus it is not necessary to perform an input operation for starting measurement. Therefore, a user who is accustomed to handling the device can quickly start the measurement only by taking a posture for collective measurement. Such an effect is suitable for a wristwatch-type wearable instrument (which is always worn) such as the biological information measurement device 10 of the present embodiment.

### (Modification)

Note that, in the flow of the biological information measurement processing described above, in a case where it is determined in step S102 that the height of the device is not within the predetermined range, the process returns to step S101 and does not proceed to step S103 until the condition is satisfied, but other processing can be performed. For example, in a case where it is determined that the height of the device is not within the predetermined range, that information may be stored in the storage unit 136, and then the process may proceed to step S103. That is, in the present modification, when the determination result of the electrode contact state determination unit 101 indicates correctness, the collective measurement execution unit 103 performs control to collectively execute the measurement of the blood pressure and the measurement of the electrocardiographic waveform by the blood pressure measurement unit 120. In this manner, it is possible to prevent that the measurement cannot be started indefinitely unless the correct posture is taken, and to store that there is a doubt in the accuracy of the measured blood pressure value and obtain at least an electrocardiographic waveform with high accuracy.

### <Others>

The description of the above-described embodiments is merely illustrative of the present invention, and the present invention is not limited to the above-described specific forms. The present invention can be variously modified and combined within the scope of the technical idea. For example, in the first embodiment, the flow of first determining whether or not the height of the device satisfies the measurement condition and then determining whether or not the contact state of the electrode satisfies the measurement condition has been described, but the order may be reversed. That is, the order of step S101 and step S103, and step S102 and step S104 may be interchanged. In short, the collective measurement can be started as long as both the condition of the height and the condition of the electrode contact state are satisfied.

In addition, in the above-described embodiment, it has been described that the guide image is displayed on the display unit 133, but the guide image may be output to an external instrument connected via the communication unit 135. In addition, the measured biological information may be streamed and transmitted to an external electronic instrument including a storage area via the communication unit 135. In addition, the configuration of the device may be omitted, and it is allowed to have a configuration without the vibration unit 137.

1. A biological information measurement device used by being worn on a wrist of a human body, the biological information measurement device comprising:
a blood pressure measurement unit configured to measure a blood pressure of the human body;
an electrocardiographic waveform measurement unit which includes a plurality of electrodes and is configured to measure an electrocardiographic waveform of the human body;
an electrode contact state detection unit configured to detect a contact state of the human body with the plurality of electrodes;
a position detection unit configured to detect a position of the device; and
a controller which controls the electrocardiographic waveform measurement unit and the blood pressure measurement unit,
wherein the controller includes
a first correctness determination unit which determines, on a basis of an output of the position detection unit, correctness as to whether or not a wrist of the human body on which the device is worn is positioned at a height within a predetermined range,
a second correctness determination unit which determines, on a basis of an output of the electrode contact state detection unit and information the baseline fluctuation of the electrocardiographic waveform and/or the posture fluctuation of the device, whether or not the human body is stably in contact with the plurality of electrodes when a determination result of the first correctness determination unit indicates correctness, and
a collective measurement control unit which automatically performs control to collectively execute blood pressure measurement of the human body by the blood pressure measurement unit and measurement of an electrocardiographic waveform of the human body by the electrocardiographic waveform measurement unit, on condition that at least a determination result of the second correctness determination unit indicates correctness.

2. The biological information measurement device according to claim 1, wherein a height within the predetermined range is set to be substantially same as a height of a heart of the human body.

3. The biological information measurement device according to claim 1, wherein the biological information measurement device is a wristwatch-type wearable device.

4. A control method of a biological information measurement device which is used by being worn on a wrist of a human body and includes
a blood pressure measurement unit configured to measure a blood pressure of the human body,
an electrocardiographic waveform measurement unit which includes a plurality of electrodes and is configured to measure an electrocardiographic waveform of the human body,
an electrode contact state detection unit configured to detect a contact state of the human body with the plurality of electrodes, and
a position detection unit configured to detect a position of the device,
the control method comprising:
a first correctness determination step which determines, on a basis of an output of the position detection unit, correctness as to whether or not a wrist of the human body on which the device is worn is positioned at a height within a predetermined range;
a second correctness determination step which determines after the first correctness determination step, on a basis of an output of the electrode contact state detection unit and information the baseline fluctuation of the electrocardiographic waveform and/or the posture fluctuation of the device, correctness as to whether or not the human body is stably in contact with the plurality of electrodes when a determination result of the first correctness determination unit indicates correctness; and
a collective measurement step which automatically collectively executes blood pressure measurement of the human body by the blood pressure measurement unit and measurement of an electrocardiographic waveform of the human body by the electrocardiographic waveform measurement unit, in a case where at least a determination result of the second correctness determination step indicates correctness.

5. The control method of the biological information measurement device according to claim 4, wherein a height within the predetermined range is set to be substantially same as a height of a heart of the human body.

6. A non-transitory computer readable medium storing a program for causing a biological information measurement device to execute each step of the control method according to claim 4.
